(19) United States
(12) Patent Application Publication
(10) Pub. No.: US 2005/0163707 A1
(43) Pub. Date: Jul. 28, 2005

(54) Cancer treatment

(76) Inventor: Gail Rowlinson-Busza, Cranmer Terrace, London (GB)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(21) Appl. No.: 10/501,568

(22) PCT Filed: Jan. 13, 2003
(86) PCT No.: PCT/GB03/00105

(30) Foreign Application Priority Data
Jan. 12, 2002 (GB) 0200657.3

(51) Int. Cl. A61K 51/00; A61K 31/704; A61K 31/337; A61K 33/24

(52) U.S. Cl. 424/1.49; 514/34; 424/649; 514/449

(57) ABSTRACT

The invention relates to a therapeutic system comprising (i) a radiolabelled antibody, which binds selectively to polymorphic epithelial mucin (PEM) such as the monoclonal antibody HMFG-1, and (ii) a chemotherapeutic agent, such as Taxotere®. The radiolabelled antibody and chemotherapeutic agent are administered in combination with one another to produce a synergistic therapeutic effect.

The present invention relates to materials and methods for the treatment of cancer. In particular, the invention relates to a therapy comprising the administration of a radiolabelled antibody, which binds selectively to polymorphic epithelial mucin (PEM) in combination with a chemotherapeutic agent.

DeNardo et al. (April 1997) P.N.A.S. USA 94 pp. 4000-4004 mention that a synergistic therapeutic effect can be obtained in a mouse model of breast cancer by administering a yttrium 90-labelled chimeric L6 antibody (⁹⁰Y-ChL6) 6 or 24 hours before Taxol® (paclitaxel) administration. However, no synergistic effect was observed when Taxol® was administered 24-27 hours before ⁹⁰Y-ChL6.

DeNardo et al. conclude as follows: ⁹⁰Y-ChL6 and Taxol® can be given in a sequence that enhances therapeutic efficacy. Over time, after injection, ⁹⁰Y-ChL6 binds to malignant cells as it circulates and unbound ⁹⁰Y-ChL6 is cleared from normal tissues. Thus a “window” in time exists when there is ongoing tumour irradiation but little concurrent normal tissue irradiation. Given in this window, Taxol®, a small molecule rapidly taken up by the tumour, enhances the therapeutic effect of ⁹⁰Y-ChL6 on targeted malignant cells. The optimum time for Taxol® administration is 6-24 hours after ⁹⁰Y-ChL6.

ChL6 consists of a human IgG constant region and the Fab′ region of murine monoclonal antibody (mAb) L6. ChL6 reacts with an integral membrane glycoprotein expressed at a high frequency on human breast, colon, ovary and lung carcinomas.

Gillies, at “Magic bullets: an update on therapeutic antibodies”, London 27 to 28 Jun. 2001, reported that CT26-EpCAM subcutaneous tumours treated with ¹²⁵I-KS-IL2 (iodine-125 labelled KS antibody IL-2 fusion) and Taxol® showed the two treatments to have a synergistic effect when the immunotherapy was given 24 hours after the chemotherapy. From this he concluded that “Optimal chemotherapeutic doses may lower tumour interstitial pressure and increase targeting of immunocytokines”, in other words the chemotherapy e.g. Taxol®, should be administered before the immunotherapeutic.

Thus, both DeNardo and Gillies suggest that the order of administration is important to the efficacy of a combined chemotherapy and immunoradiotherapy. However they disagree on the most useful order of administration for the chemotherapeutic and immunotherapeutic agents.

The search for anti-cancer agents and methods of treatment is ongoing and intense. The present invention seeks to provide further agents and methods for the treatment of cancers.

### SUMMARY OF THE INVENTION

The inventor has discovered that a synergistic tumouricidal effect can be obtained by means of a combined treatment with a radiolabelled antibody that binds selectively to polymorphic epithelial mucin (PEM), and a chemotherapeutic agent.

PEM is a component of the human milk fat globule. PEM is expressed by cells in several body tissues and is also found in urine. Significantly, PEM is known to be expressed in epithelial cancer cells, notably in ovarian, gastric, colorectal and pancreatic cancer cells.

The preferred chemotherapeutic agent is the antineoplastic agent Taxotere® (Docetaxel), which is a semi-synthetic analogue of Taxol®. For an overview of Taxotere® see J L Fabre et al. (1995) Drugs Future, 20, pp. 464-471. For synthesis and structure see M Colin et al. U.S. Pat. No. 4,924,012 (to Rhône-Poulenc Sante). For anti cancer activity see Riou et al. (1992) Biochem. Biophys Res. Commun. 187, pp. 164-170. Taxotere® is available commercially from Rhône-Poulenc Rorer. Other preferred chemotherapeutic agents are: Cisplatin (Faulding), Cyclophosphamide (Pharmacia and Upjohn), Vincristine (Faulding) and Gemcitabine (Lilly).

Monoclonal antibodies that will bind to PEM are already known, but in any case, with today's techniques in relation to monoclonal antibody technology, antibodies can be prepared to most antigens. Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982) and “Antibody Engineering, A Practical Approach”, J McCafferty et al., ed. (IRL Press, 1996).

WO 01/74905 discloses antibodies that bind selectively to PEM and are useful in accordance with the present invention.

Preferably, the antibody is HMFG-1, which is available from Imperial Cancer Research Fund, England. More preferably the antibody is a humanised HMFG-1. Such antibodies are disclosed in WO 92/04380.

HMFG antibodies are raised against human milk fat globule (HMFG), in a delipidated state (see Taylor-Papadimitriou et al., (1981), Int. J. Cancer 28 pp. 17-21 and Gendler et al., (1988), J. Biol. Chem. 236 pp. 12820-12823).

HMFG-1 monoclonal antibodies bind to a particular component of HMFG, namely polymorphic epithelial mucin REM). Binding is thought to involve the amino acid sequence APDTR within the twenty amino acid tandem repeats of the muc-1 gene product.

By ‘humanised antibody’ we include monoclonal antibodies having at least one chain wherein the framework regions are predominantly derived from a first, acceptor monoclonal antibody of human origin and at least one complementarity-determining region (CDR) is derived from a second, donor monoclonal antibody that may be of human or non-human origin, for example it may be a murine monoclonal antibody.

Preferably both chains of the humanised monoclonal antibody CDRs are grafted from a donor monoclonal antibody having specificity for PEM.

Advantageously, the CDR-grafted (i.e. humanised) chain comprises two or all three CDRs derived from a donor antibody having specificity for PEM.

Conveniently, the humanised monoclonal antibody comprises only human framework residues and CDRs from a donor antibody having specificity for PEM.

However, it will be appreciated by those skilled in the art that in order to maintain and optimise the specificity of the humanised antibody it may be necessary to alter one or more residues in the framework regions such that they correspond to equivalent residues in the donor antibody.

Conveniently, the framework regions of the humanised antibody are derived from a human IgG monoclonal antibody.

Methods of making humanised monoclonal antibodies are well-known in the art, for example see Jones et al. (1986) Nature 321 pp. 522-525, Riechmann et al. (1988) Nature 322 pp. 323-327, Verhoeyen et al. (1988) Science 239 pp. 1534-1536 and EP 239 400 (to Winter).

By “antibody” we include antibody fragments and antigen binding molecules. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the V_{H} and V_{L} partner domains are linked via a flexible oligopeptide.

The advantages of using antibody fragments, rather than whole antibodies, are several-fold. The smaller size of the fragments may lead to improved pharmacological properties, such as better penetration of solid tissue. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining sites.

By “in combination with one another” regarding the antibody and chemotherapeutic agent treatments we include the meaning not only that the antibody and chemotherapeutic agents are administered simultaneously, but also that they are administered separately and sequentially.

Preferably the antibody and chemotherapeutic agents are administered between 0 and 24 hours apart with either the antibody or the chemotherapeutic being administered first.

By “binds selectively” we include the meaning that the antibodies in question will specifically bind cells displaying PEM on their surface and will not bind to those cells not displaying PEM.

By “treatment” we include the meanings that the tumour size is reduced and/or further tumour growth is retarded and/or prevented and/or the tumour is killed.

Examples embodying an aspect of the invention will now be described with reference to the following figures in which:

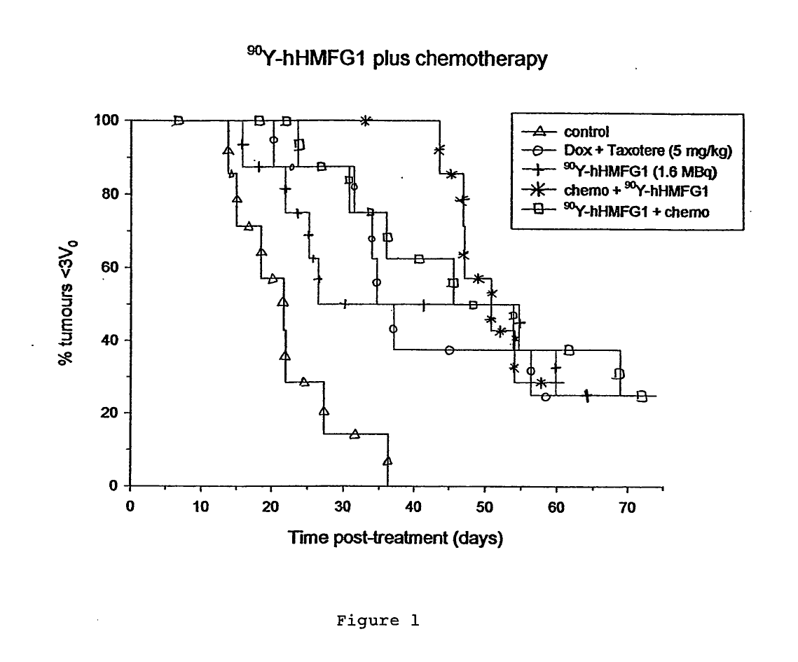

FIG. 1 shows the effect of various treatments on tumour volume in a human derived bladder cancer cell line subcutaneously implanted on a mouse.

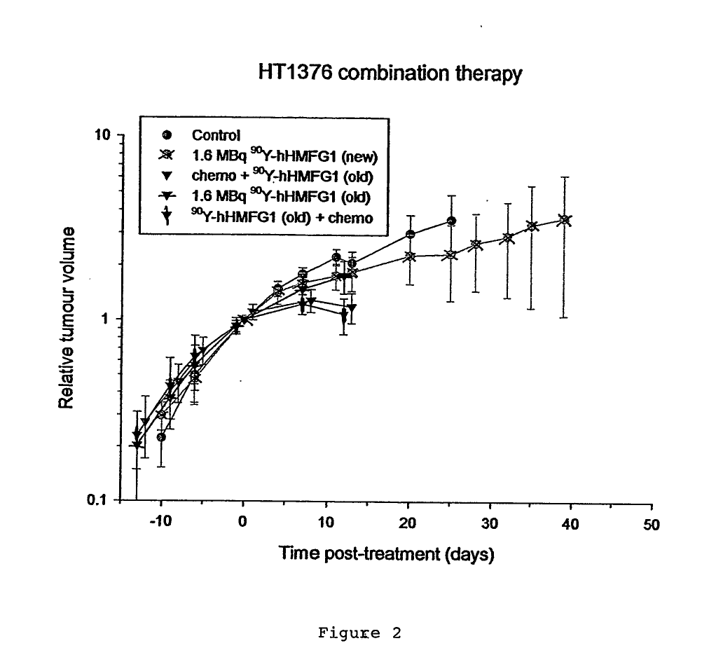

FIG. 2 shows the effect on tumour tripling times of various treatments.

### EXAMPLE 1

### Combination Therapy Increases Tumour-Tripling Times significantly

Materials & Methods

Cell Lines

The human bladder tumour cell line, HT1376, and the human colon tumour cell line HT29 expressing polymorphic epithelial mucin (PEM) were cultured in RPMI 1640 tissue culture medium containing 100 U·ml⁻¹ penicillin and 100 μg·ml⁻¹ streptomycin, supplemented with 10% foetal calf serum in a humidified atmosphere of 5% carbon dioxide in air. HT1376 is a human bladder carcinoma cell line obtained from the European Collection of Animal Cell Cultures (ECACC no. 87032402). HT29 is human colon carcinoma cell line obtained from European Collection of Animal Cell Cultures (ECACC no. 91072201).

Antibody

The fully humanised version of the anti-PEM antibody, HMFG1, was produced by Lonza, Slough, UK. This humanised HMFG1 (hHMFG1) was conjugated with the chelating agent CITC-DPTA by BioInvent, Sweden.

Radiolabelling

CITC-DTPA-conjugated hHMFG1 was radiolabelled with ⁹⁰Y in acetate buffer (pH 5.5) at room temperature for 30 minutes. Disodium EDTA was added to the reaction mixture such that the final EDTA concentration was 5 mM and left to stand at room temperature for approximately 10 min. The radiolabelled protein was then purified by size exclusion chromatography and the protein-containing fractions pooled.

Animal Model

Mice

Female MF1 athymic nude (nu/nu) mice were used throughout these studies. The mice were bred at the Biological Research Facility of St. George's Hospital Medical School and were housed in sterile filter cages and maintained on irradiated diet and sterile water. Tumours were established by subcutaneous injection of 5×10⁶ cells in the right flank.

Combination Therapy

Stock solutions of drugs: Taxotere (Aventis), Cisplatin (Faulding), Cyclophosphamide (Pharmacia and Upjohn), Vincristine (Faulding) and Gemcitabine (Lilly) were diluted in saline and injected intravenously into tumour bearing nude mice via a lateral tail vein. Humanised HMFG1 anti-PEM antibody (previously conjugated with the chelating agent CITC-DTPA) was radiolabelled with yttrium-90 (⁹⁰Y) to a specific activity of approximately 1-2 MBq per 10 μg. Mice received approximately 10 μg of protein by intravenous injection.

For combination therapy, the drug and radioimmunotherapy (RIT) were given by sequential injections into the two lateral tail veins. The drug was given either 24 h before or 24 h after the RIT. In the case of Cisplatin, this was also given at the same time as the RIT. Control mice were untreated.

Tumour Therapy Schedule

Approximately three weeks after tumour inoculation, when the tumours were around 0.2 cm³ in volume (7-8 mm in diameter), mice were divided into treatment groups of 7-8 mice each. The tumour volumes in each group at the time of treatment were not significantly different. Mice were injected with various doses of ⁹⁰Y-labelled hHMFG1 radioimmunotherapy (10-20 μg, 1.2-2.0 MBq) either alone or in combination with the test drug given either 24 h before or 24 h after radioimmunotherapy, or with chemotherapy or chemotherapy vehicle alone. One group of mice was left untreated as a control on some occasions when experimental animals were treated. Tumour diameters (d₁, d₂ and d₃) were measured twice weekly in three orthogonal directions using a vernier calliper and the tumour volume (v) calculated according to the formula for an ellipsoid:

$$v = \frac{π}{6} ( {d}_{1} \cdot {d}_{2} \cdot {d}_{3} )$$

Tumour measurement commenced one week before treatment and continued until the tumours had at least tripled in volume. Relative tumour volume (the volume of each tumour divided by the tumour volume on the day of treatment) was calculated to minimise the effect of variation in treatment volume of the individual tumours. The end-point was defined as time for the relative tumour volume to reach 3.

Statistics

The Wilcoxon rank sum test was used to compare the groups of mice receiving the various treatment protocols. A p-value <0.05 was considered to be significant.

Results

Volume Tripling Times (Days)

⁹⁰Y-hHMFG1 Plus Chemotherapy

TABLE 1

| Treatment | Median | T-C |
|---|---|---|
| Control (18-19) | 17.9 | |
| Taxotere ® 10 mg/kg (15) | 43.8 | 25.9 |
| 1.2 MBq ⁹⁰Y-hHMFG1 | 23.4 | 5.5 |
| 2.0 MBq ⁹⁰Y-hHMFG1 | 46.5 | 28.6 |
| 1.2 MBq + Taxotere ® | 73.3 | 55.4 |
| Control (25-26) | 19.0 | |
| Taxotere ® 10 mg/kg (27-31) | 41.2 | 22.2 |
| Dox 10 mg/kg (20-22) | 52.9 | 33.9 |
| Taxol ® 10 mg/kg (23) | 30.2 | 11.2 |
| Control (76-79) | 21.7 | |
| 1.6 MBq ⁹⁰Y-hHMFG1 | 40.6 | 18.9 |
| Tax + dox 5 mg/kg each | 35.9 | 14.2 |
| 1.6 MBq + chemo | 49.8 | 28.1 |
| Chemo + 1.6 MBq (80) | 50.8 | 29.1 |

TABLE 2

| | | |
|---|---|---|
| Treatment | Median | T-C |

HT1376 Taxotere

| | | |
|---|---|---|
| Observer 1 | | |
| Control | 21.9 | |
| 1.2 MBq ⁹⁰Y-hHMFG1 | 23.8 | 1.9 |
| 10 mg/kg Taxotere | 43.5 | 21.6 |
| Taxotere + 1.2 MBq | 49.5 | 27.6 |
| 1.2 MBq + Taxotere | 51.7 | 29.8 |

HT1376 Cisplatin

| | | |
|---|---|---|
| Observer 1 | | |
| Control | 21.9 | |
| 1.2 MBq ⁹⁰Y-hHMFG1 | 22.9 | 1.0 |
| 10 mg/kg Cisplatin | 28.8 | 6.9 |
| Cisplatin + 1.2 MBq (−24 h) | 65.9 | 44.0 |
| 10 mg/kg + 1.2 MBq (0 h) | 58.4 | 36.5 |
| 1.2 MBq + Cisplatin (+24 h) | 27.4 | 15.5 |
| Control | 21.9 | |
| 1.4 MBq ⁹⁰Y-hHMFG1 | 30.9 | 9.0 |
| 10 mg/kg Cisplatin | 28.8 | 6.9 |
| 10 mg/kg + 1.4 MBq (−24 h) | 46.6 | 24.7 |
| Control | 21.9 | |
| 1.4 MBq ⁹⁰Y-hHMFG1 | 30.9 | 9.0 |
| 2 mg/kg Cisplatin | 28.9 | 7.0 |
| 2 mg/kg + 1.4 MBq (0 h) | 37.5 | 15.6 |

HT29 gemcitabine

| | | |
|---|---|---|
| Observer 1 | | |
| Control | 8.8 | |
| 1.4 MBq ⁹⁰Y-hHMFG1 | 15.7 | 6.9 |
| 240 mg/kg gemcitabine | 15.4 | 6.6 |
| Gemcitabine + 1.4 MBq | 15.8 | 7.0 |
| 1.4 MBq + gemcitabine | 19.0 | 10.2 |
| Observer 2 | | |
| Control | 9.5 | |
| 1.4 MBq ⁹⁰Y-hHMFG1 | 14.4 | 4.9 |
| 240 mg/kg gemcitabine | 12.8 | 3.3 |
| Gemcitabine + 1.4 MBq | 14.6 | 5.1 |
| 1.4 MBq + gemcitabine | 12.2 | 2.7 |

HT29 cyclophosphamide

| | | |
|---|---|---|
| Observer 1 | | |
| Control | 10.2 | |
| 1.4 MBq ⁹⁰Y-hHMFG1 | 11.8 | 1.6 |
| 200 mg/kg cyclo | 15.5 | 5.3 |
| Cyclo + 1.4 MBq | 13.0 | 2.8 |
| 1.4 MBq + cyclo | 17.4 | 7.2 |
| Observer 2 | | |
| Control | 10.7 | |
| 1.4 MBq ⁹⁰Y-hHMFG1 | 13.2 | 2.5 |
| 200 mg/kg cyclo | 14.1 | 3.4 |
| Cyclo + 1.4 MBq | 13.8 | 3.1 |
| 1.4 MBq + cyclo | 17.5 | 6.8 |

HT29 vincristine

| | | |
|---|---|---|
| Observer 1 | | |
| Control | 10.2 | |
| 1.4 MBq ⁹⁰Y-hHMFG1 | 11.8 | 1.6 |
| 2 mg/kg vincristine | 13.4 | 3.2 |
| Vincristine + 1.4 MBq | 15.6 | 5.4 |
| 1.4 MBq + vincristine | 16.4 | 6.2 |
| Observer 2 | | |
| Control | 10.7 | |
| 1.4 MBq ⁹⁰Y-hHMFG1 | 13.2 | 2.5 |
| 2 mg/kg vincristine | 14.7 | 4.0 |
| Vincristine + 1.4 MBq | 14.2 | 3.5 |
| 1.4 MBq + vincristine | 16.6 | 5.9 |

The tables summarise the treatments given and the results obtained and show the median tumour volume tripling times for HT1376 bladder tumour xenografts treated with Taxotere or Cisplatin and for HT29 colon tumour xenografts treated with Gemcitabine, Cyclophosphamide, or Vincristine. The second column is the treated minus control tripling time, i.e. the advantage of the drug or drug combination over untreated tumours. Hence, the larger the T-C value, the better the therapeutic effect obtained. Two sets of data are presented, due to tumour measurements being taken by two observers. FIGS. 1 & 2 represent graphically a selection of the results obtained.

From Table 1 it is clear that a combination of 1.2 MBq ⁹⁰Y-hHMFG1 and Taxotere® increased the tumour tripling time more than twice as much as Taxotere® on its own and ten times as much as the same dose of ⁹⁰Y-hHMFG1 on its own. In fact, the contribution of radiolabelled antibody and Taxotere® was almost twice as effective (T-C=55.4) as the additive effect of the individual treatments (T-C=5.5+25.9=31.4).

Table 1 also shows that the sequence of treatment with ⁹⁰Y-hHMFG1 and Taxotere® does not alter the effectiveness of the treatment significantly. Administration of 1.6 MBq ⁹⁰Y-hHMFG1 before Taxotere® gave a T-C value of 28.1 whereas administration in the reverse order gave a T-C value of 29.1.

From Table 2 it is clear that for example a combination of 1.2 MBq ⁹⁰Y-hHMFG1 and Taxotere® increased the tumour tripling time more than 50% over Taxotere® on its own and fifteen times as much as the same dose of ⁹⁰Y-hHMFG1 on its own. The contribution of radiolabelled antibody and Taxotere® was 25% more than the additive effect of the individual treatments (T-C=29.8 in combination, T-C=1.9+21.6=23.5).

Table 2 also shows that the sequence of treatment with ⁹⁰Y-hHMFG1 and Taxotere® does not alter the effectiveness of the treatment significantly. Administration of 1.2 MBq ⁹⁰Y-hHWFG1 before Taxotere® gave a T-C value of 29.8 whereas administration in the reverse order gave a T-C value of 27.6.

Table 2 also shows that Cisplatin demonstrates a synergistic effect no matter the order of administering the radioimmunotherapy and the drug. For example, the 1.2 MBq dose gives a T-C value of 44, 36.5 and 15.5 over the different dosage regimes, the smallest of which is double the combined addition of the drugs administered individually at a T-C value of 7.9. The most effective treatment was to administer the drug 24 hours before the radioimuunotherapy. Gemcitabine also showed an overall increase in T-C values when the drug and radioimmunotherapy where given in combination.

Cyclophosphamide and vincristine were both most effective when given after the radioimmunotherapy. In that treatment they showed a synergistic effect over the mere additive effect of the drugs administered individually. Vincristine is also effective when administered before the radioimmunotherapy although to a slightly lesser extent.

Overall, the data shows that the combination therapy is more effective than either treatment administered alone. The effects are of differing amounts but represent a synergistic effect (i.e. more than additive).

### EXAMPLE 2

### Use of Combination Therapy in Tumour Treatment

The combination therapy experimentally tested on mouse tumours in example 1 can be applied to use in the treatment of human tumours.

Treatment of human tumours requires administration of the standard clinical chemotherapy dose in mg/m² (mg/m² is calculated approximately by multiplying mg/kg by 230) for the chemotherapeutic agent being used. The standard clinical dose for a particular patient can easily be calculated based on that patient's specific circumstances and would form part of the day to day activities of the skilled person.

The radiolabelled antibody would preferably be administered at an initially low dose e.g. 37 to 185 Mbeq (1 to 5 milliCuries) of radiation. It is envisaged that this initially low dose of radioimmunotherapy can be raised in subsequent doses, dependent on the individual requirements of the patient. Higher doses are envisaged to be administered in amounts such that up to 400 Mbeq of radioimmunotherapy can safely reach the target tumour.

Maraveyas et al. (1995) Cancer Research; 55; pp 1020-102 (incorporated herein by reference), discloses that 30% of intraperitoneal radiolabelled antibody reaches the target tumour site. Hence, it is well within the knowledge of a skilled person to apply the teaching of Maraveyas to tailoring the most appropriate radioimmunotherapy dose, based on the amount of radiation to reach the target tumour and the weight (and/or surface area) of the patient, in conjunction with any other relevant factors.

The time between administration of the chemotherapeutic agent and the radioimmunotherapy is preferably between 0 and 24 hours, with either the chemotherapeutic or the radioimmunotherapy being administered first. It is well within the skilled person's capabilities to construct a schedule of times for administering the chemotherapeutic and radioimmuunotherapeutic based on the needs of the patient and availability of appropriate resources. The combination therapy will be administered in a course of treatment. The exact frequency of treatment administration within the course and length of the course as a whole will depend upon the particular chemotherapeutic agent being used and the circumstances of the individual patient. It is entirely within the scope of a skilled person's abilities to be able to determine the appropriate length and frequency of treatment.

1. A therapeutic system for the treatment of tumours comprising a combination of component (i) a radiolabelled antibody and component (ii) a chemotherapeutic agent, wherein the antibody binds selectively to polymorphic epithelial mucin (PEM); the components (i) and (ii) being provided for use in the treatment of a tumour wherein the radiolabelled antibody and a chemotherapeutic agent are administered in combination with one another.

2. A therapeutic system for use as claimed in claim 1 wherein the antibody treatment precedes treatment with the chemotherapeutic agent.

3. A therapeutic system for use as claimed in claim 1 wherein the chemotherapeutic agent treatment precedes treatment with the antibody.

4. A therapeutic system as claimed in any of claims 1 to 3 wherein the antibody is humanised.

5. A therapeutic system as claimed in claim 4 wherein the antibody is HMFG-1.

6. A therapeutic system as claimed in any preceding claim wherein the chemotherapeutic agent is selected from at least one of docetaxel, paclitaxel, doxorubicin and cisplatin.

7. A therapeutic system for use as claimed in claim 6 wherein the chemotherapeutic agent is docetaxel.

8. A therapeutic system as claimed in any of claims 1 to 5 wherein the chemotherapeutic agent is selected from at least one of gemcitabine, cyclophosphamide and vincristine.

9. A therapeutic system as claimed in any previous claim wherein the tumour is associated with at least one of the following disorders: breast cancer, ovarian cancer, lung cancer, gastric cancer, bladder cancer and squamous cell carcinoma, such as head and neck cancer.

10. A method of treating a tumour comprising exposing the tumour to a combination of component (i) a radiolabelled antibody and component (ii) a chemotherapeutic agent, wherein the antibody binds selectively to polymorphic epithelial mucin (PEM).

11. A method as claimed in claim 10 wherein the antibody treatment precedes treatment with the chemotherapeutic agent.

12. A method as claimed in claim 10 wherein the chemotherapeutic agent treatment precedes treatment with the antibody.

13. A method as claimed in any of claims 10 to 12 wherein the antibody is humanised.

14. A method as claimed in claim 13 wherein the antibody is HMFG-1.

15. A method as claimed in any of claims 10 to 14 wherein the chemotherapeutic agent is selected from at least one of docetaxel, paclitaxel, doxorubicin and cisplatin.

16. A method as claimed in claim 15 wherein the chemotherapeutic agent is docetaxel.

17. A method as claimed in any of claims 10 to 14 wherein the chemotherapeutic agent is selected from at least one of gemcitabine, cyclophosphamide and vincristine.

18. A method as claimed in any of claims 10 to 17 wherein the tumour is associated with at least one of the following disorders: breast cancer, ovarian cancer, lung cancer, gastric cancer, bladder cancer and squamous cell carcinoma, such as head and neck cancer.

19. Use of a combination of component (i) a radiolabelled antibody and component (ii) a chemotherapeutic agent, wherein the antibody binds selectively to polymorphic epithelial mucin (PEM) in the manufacture of a therapeutic system for the treatment of cancer.

20. A use as claimed in claim 19 wherein the antibody is humanised.

21. A use as claimed in claim 20 wherein the antibody is HMFG-1.

22. A use as claimed in any of claims 19 to 21 wherein the chemotherapeutic agent is selected from at least one of docetaxel, paclitaxel, doxorubicin and cisplatin.

23. A use as claimed in claim 22 wherein the chemotherapeutic agent is docetaxel.

24. A use as claimed in any of claims 19 to 21 wherein the chemotherapeutic agent is selected from at least one of gemcitabine, cyclophosphamide and vincristine.

25. A use as claimed in any of claims 19 to 24 wherein the tumour is associated with at least one of the following disorders: breast cancer, ovarian cancer, lung cancer, gastric cancer, bladder cancer and squamous cell carcinoma, such as head and neck cancer.

26. A therapeutic system substantially as described herein with reference to one or more of the examples.

27. A method of treatment substantially as described herein with reference to one or more examples.

28. Use of a combination of component (i) a radiolabelled antibody and component (ii) a chemotherapeutic agent, wherein the antibody binds selectively to polymorphic epithelial mucin (PEM) in the manufacture of a therapeutic system for the treatment of cancer substantially as described herein with reference to one or more examples.
